(19) United States
(12) Patent Application Publication
(10) Pub. No.: US 2018/0368907 A1
(43) Pub. Date: Dec. 27, 2018

(54) TREATMENT INSTRUMENT AND TREATMENT SYSTEM

(71) Applicant: OLYMPUS CORPORATION, Tokyo (JP)

(72) Inventor: Tsunetaka AKAGANE, Hachioji-shi (JP)

(73) Assignee: OLYMPUS CORPORATION, Tokyo (JP)

(21) Appl. No.: 16/047,561

(22) Filed: Jul. 27, 2018

Related U.S. Application Data
(63) Continuation of application No. PCT/JP2016/052714, filed on Jan. 29, 2016.

(51) Int. Cl. A61B 18/12; A61B 18/08

(52) U.S. Cl. CPC A61B 18/1206; A61B 2017/00084; A61B 18/08

(57) ABSTRACT

A treatment instrument includes a treatment section including an electric heating member. The electric heating member is formed by mixture of a conductive material into a nonconductive material and includes one end and the other end. Resistance heating is caused in the electric heating member when a current is applied between the one end and the other end of the electric heating member.

### CROSS-REFERENCE TO RELATED APPLICATIONS

This application is a Continuation Application of PCT Application No. PCT/JP2016/052714, filed Jan. 29, 2016, the entire contents of all of which are incorporated herein by reference.

### BACKGROUND OF THE INVENTION

### 1. Field of the Invention

The present invention relates to a treatment instrument and a treatment system which is configured to treat a treatment object using heat generated in a treatment section.

2 Description of the Related Art

For example, Jpn. Pat. Appln. KOKAI Publication No. 2005-137679 discloses a treatment instrument which is used in such a manner that a treatment section thereof is inserted into a body cavity or the like. The treatment section of the treatment instrument includes a heating element which generates heat upon energization, such as a thin-film resistance heating element, a thick-film resistance heating element, a ceramic heater, or a PTC heater. For a thin-film resistance heating element, a pattern of an electric heating wire is formed in a ceramic material or a metal substrate by a thin-film forming method. For a thick-film resistance heating element, a pattern of an electric heating wire is formed in a ceramic material or a metal substrate by a thick-film forming method.

In order to safely raise a temperature of an electric heating wire of a heat generation section, it is necessary to increase electric resistance of an electric heating wire. For example, an electric heating wire is made finer and longer, that is, a path length from one end to the other end of an electric heating wire is made longer by formation of the electric heating wire in a sinuous shape as a specific example, to increase electric resistance of an electric heating wire.

### BRIEF SUMMARY OF THE INVENTION

According to an aspect of the invention, there is provided a treatment instrument includes a treatment section including an electric heating member which is formed by mixture of a conductive material into a nonconductive material and includes one end and the other end, wherein resistance heating is caused in the electric heating member when a current is applied between the one end and the other end of the electric heating member.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view showing a treatment system according to a first embodiment.

FIG. 2A is a schematic cross-sectional view showing an electric heating member and a heat transmission plate which are included in a treatment section of a treatment instrument in the treatment system according to the first embodiment.

FIG. 2B is a schematic cross-sectional view showing the electric heating member, an insulation layer, and the heat transmission plate which are included in the treatment section of the treatment instrument in the treatment system according to the first embodiment.

FIG. 3A is a schematic view of the electric heating member which is included in the treatment section of the treatment instrument in the treatment system according to the first embodiment.

FIG. 3B is a schematic view of an electric heating member which is included in a treatment section of the treatment instrument in the treatment system according to a modification of the first embodiment.

FIG. 4 is a schematic graph showing a relationship between a temperature and an electric resistance value of the electric heating member which is included in the treatment section of the treatment instrument in the treatment system according to the first embodiment.

FIG. 5A is a schematic perspective view showing the treatment section and a portion of a housing in the neighborhood of the treatment section of the treatment instrument, in the treatment system according to the first embodiment.

FIG. 5B is a schematic cross-sectional view taken along a line 5B-5B in FIG. 5A.

FIG. 6 is a schematic view showing a treatment system according to a second embodiment.

FIG. 7A is a schematic cross-sectional view of a treatment section of a treatment instrument in the treatment system according to the second embodiment, as taken along a line 7A-7A in FIG. 6.

FIG. 7B is a schematic cross-sectional view of a treatment section of the treatment instrument in the treatment system according to a modification of the second embodiment, as taken along a line 7A-7A in FIG. 6.

FIG. 8 is a schematic view showing a treatment system according to a third embodiment.

FIG. 9 is a schematic cross-sectional view taken along a line IX-IX in FIG. 8.

### DETAILED DESCRIPTION OF THE INVENTION

Below, embodiments for carrying out the present invention will be described with reference to the drawings.

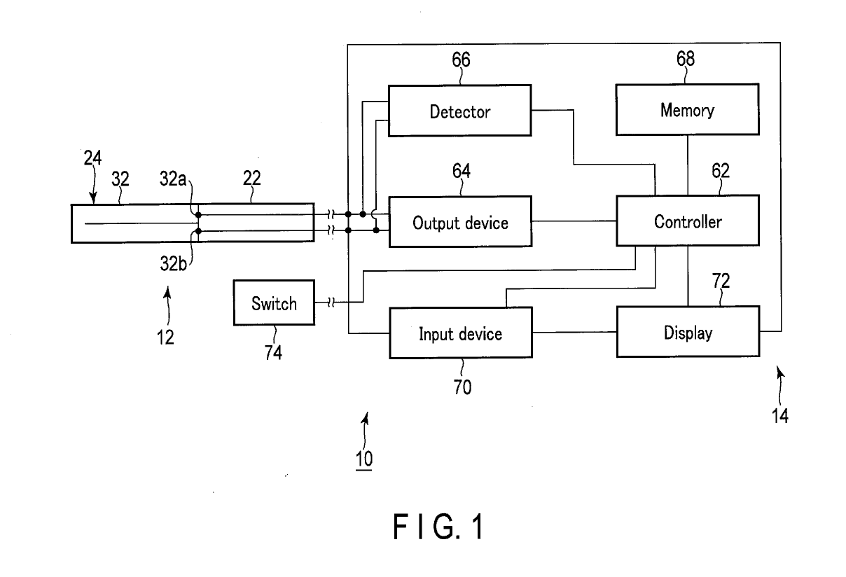

First, a first embodiment will be described with reference to FIGS. 1 to 5B.

As shown in FIG. 1, a treatment system 10 according to the present embodiment includes a treatment instrument 12 and an energy source 14 which is configured to supply energy to the treatment instrument 12.

The treatment instrument 12 includes a housing 22 which is configured to be gripped by an operator and has an electrically-insulating property, and a treatment section 24 which is configured to be brought into contact with a treatment object, to provide treatment.

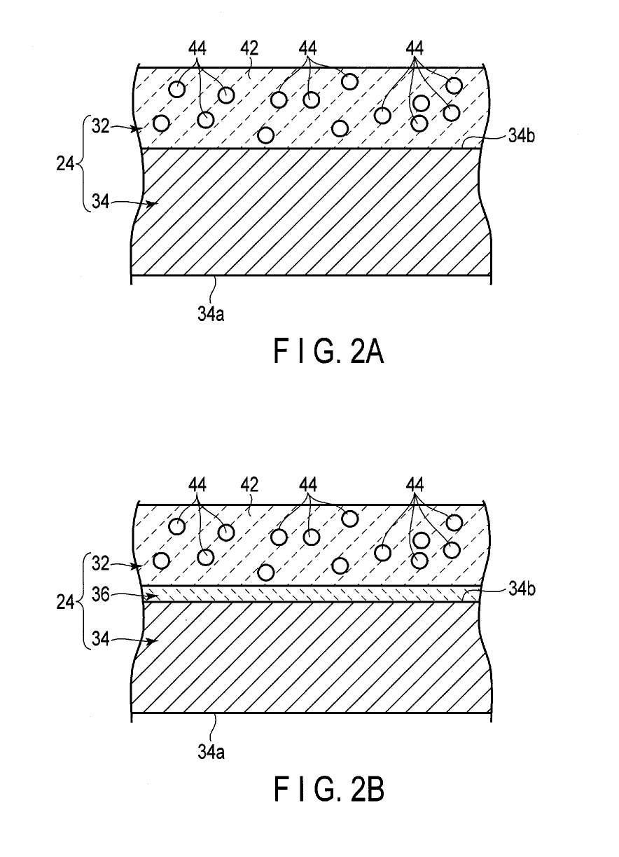

As shown in FIGS. 2A and 2B, the treatment section 24 includes an electric heating member (heating element) 32 in which resistance heating (heat generation) is caused in the electric heating member 32 when a current is applied to the electric heating member 32, and a heat transmission body (heat transmission member) 34 which is configured to transmit heat of the electric heating member 32.

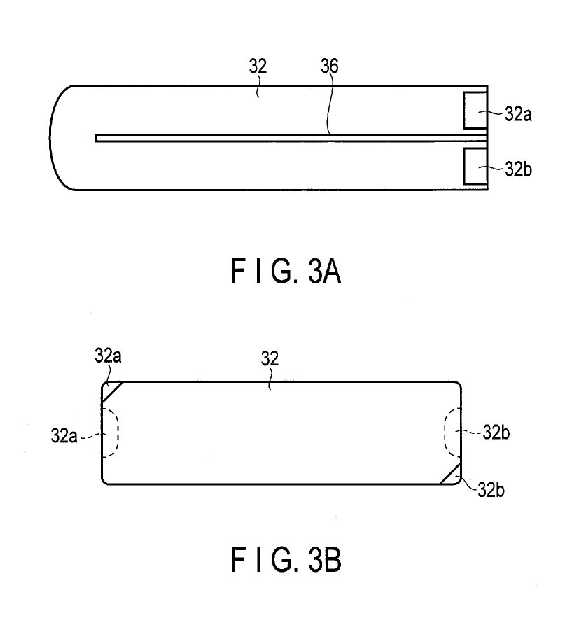

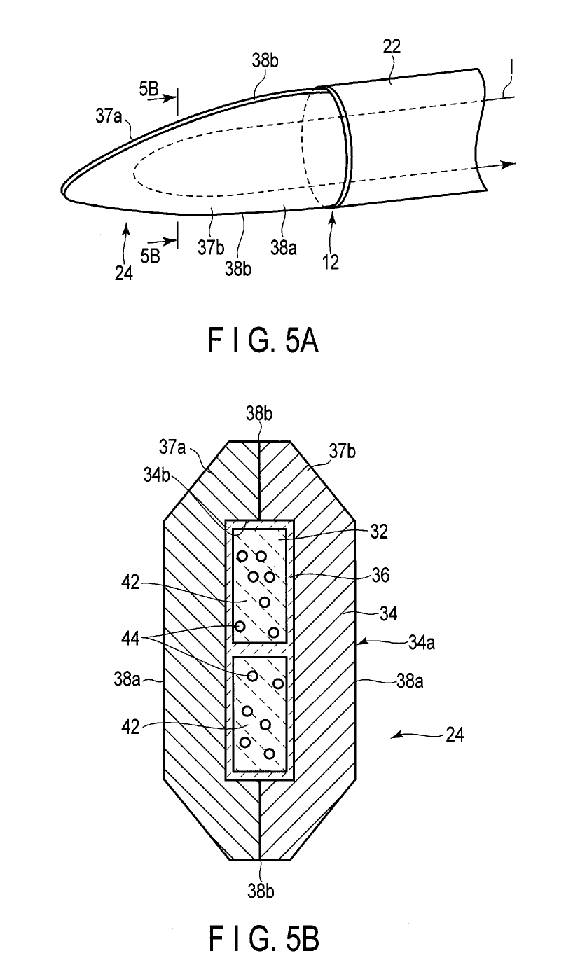

Now, it should be noted that the treatment section 24 (refer to FIG. 5A) which is to be inserted into a body cavity is required to be formed in an extremely small size such a size with a width of approximately several millimeters and a length in a range between several millimeters and several dozen millimeters, for example. For this reason, it can be said that it is difficult to directly measure a temperature of the treatment section 24. In the electric heating member 32 in the present embodiment, an electric resistance value R between terminals 32a and 32b of the electric heating member 32 changes in accordance with a heat-generation temperature T of the electric heating member 32. Accordingly, the electric heating member 32 in the present embodiment is configured such that the temperature T can be estimated based on the electric resistance value (measured value) R between the terminals 32a and 32b (refer to FIGS. 3A and 3B) which will be described later.

The electric heating member 32 according to the present embodiment is required to have an electric resistance value which does not become constant or substantially constant even with a rise of temperature, but which increases as a temperature rises. In other words, a temperature coefficient of resistance of the electric heating member 32 according to the present embodiment is required to be high. Further, it is presumed that in providing treatment, a certain temperature (an appropriate temperature in a range between approximately 200° C. and 300° C., for example) is suitable for coagulation or incision of living-body tissue. For this reason, it is preferable that an electric resistance value of the electric heating member 32 changes in such a manner that an inclination (dR/dT) of a change in an electric resistance value per unit temperature (electric resistance value/temperature) increases as a temperature rises from an ordinary temperature (room temperature).

Additionally, it is preferable that a temperature of the electric heating member 32 can be raised to a temperature (approximately 350° C., for example) exceeding the above-described certain temperature in a few seconds, and a specific volume resistivity of the electric heating member 32 is required to be high.

Then, as yet, there exists no material (metal material) which has a high temperature coefficient of resistance and a high specific volume resistivity. For example, with regard to a nichrome wire which is widely used as an electric heating wire, although it can be said that specific volume resistivity thereof is high (specific volume resistivity thereof is supposed to be approximately 108×10⁻⁸[Ω·m] at 20° C. and to be approximately 110×10⁻⁸[Ω·m] at appropriately 300° C. in a certain example), a temperature coefficient of resistance thereof is 0.09×10⁻³, and so it is hard to say that specific volume resistivity thereof is higher than that of the other metal materials. For this reason, it is difficult to accurately estimate a temperature of a nichrome wire from a measured electric resistance value even by measuring a relationship between an electric resistance value between terminals of a nichrome wire and a temperature. Therefore, it is difficult to control a temperature of a nichrome wire in accordance with an electric resistance value between terminals of a nichrome wire.

The electric heating member 32 according to the present embodiment is formed by mixture of a conductive material 44 into a nonconductive material 42. More specifically, the electric heating member 32 is formed as a composite member in which the conductive material 44 is mixed into the nonconductive material 42 and is dispersed in the nonconductive material 42.

For the nonconductive material 42, a material which does not have electrical conductivity and is heat-resistant, is used. For the nonconductive material 42, ceramic coating material is used, for example. As ceramic coating material, a glass-based material which exhibits resistance to heat at approximately 900° C., for example, and has an electrically-insulating property, more specifically, methyl silicone, can be used. It is noted that not an electrically-insulating material, but a semiconductor material may be used as the nonconductive material 42.

For the conductive material 44, a metal material having conductivity is used. As described above, it is preferable that a material having a temperature coefficient of resistance which becomes relatively high when the electric heating member 32 is formed, is used as the conductive material 44. While silver powder (particulate material) is used here as a metal material which has a relatively high temperature coefficient of resistance for the conductive material 44, gold powder, copper powder, or the other metal materials, for example, can be used as appropriate. Also, for the conductive material 44, a mixture of plural kinds of metal materials may be used. Since the electric heating member 32 is formed as a composite member, there is no specific limitation to the kind of metal material so far as it can improve operability at a desired temperature, such as approximately 300° C., for example, details of which will be given later. Also, while particulate material is used here for the conductive material 44, an appropriate particle size and an appropriate shape are employed therefor.

The electric heating member 32 is formed so as to provide high specific volume resistivity and is formed as a heating element which generates heat when a current is applied between the terminals 32a and 32b. It is possible to adjust specific volume resistivity of the electric heating member 32 by mixing the nonconductive material 42 providing specific volume resistivity which is higher than that of a conductor such as a metal material, with the conductive material 44 which provides low specific volume resistivity such as silver, for example, which will be later described. Silver is supposed to provide specific volume resistivity of 1.62×10⁻⁸[Ω·m] at 20° C. and provide specific volume resistivity of approximately 3.34×10⁻⁸[Ω·m] at approximately 300° C. in a certain example. For this reason, it is much more difficult to cause silver to generate heat by flowing of a current to silver than to cause a nichrome wire to generate heat by flowing of a current to the nichrome wire. In a case where an electric heating member is formed using silver alone without the use of the nonconductive material 42, for example, there arises a need of forming the electric heating member 32 in a fine and long shape or unduly lengthening a path length of the electric heating member 32. In the meantime, the electric heating member 32 of a composite member is formed by mixture of silver which is provided as the conductive material 44 into the nonconductive material 42, so that a value of specific volume resistivity of the electric heating member 32 is made close to, or slightly higher than, that of a nichrome wire.

It is noted that a temperature coefficient of resistance of silver, which is 4.1×10⁻³, is higher than that of a nichrome wire. Accordingly, as a temperature rises, an electric resistance value of silver changes to a greater extent than that of a nichrome wire.

As shown in FIG. 3A, the electric heating member 32 includes one end (first terminal) 32a and the other end (second terminal) 32b. Described here is an example in which the electric heating member 32 is formed in an almost U shape, as one example. A clearance is provided or a part of an insulation layer 36 (refer to FIG. 2B) which will be described later is placed between the one end 32a and the other end 32b. The electric heating member 32 may have any width that allows the electric heating member 32 to be placed in the treatment section 24, such as a width of approximately one millimeter, for example. Also, the electric heating member 32 may have any thickness that allows the electric heating member 32 to be placed in the treatment section 24, and the electric heating member 32 is not necessarily required to be in a form of a thin film.

Owing to resistance heating which is caused when a current is fed to flow from the one end 32a to the other end 32b of the electric heating member 32, heat can be generated in the electric heating member 32. A magnitude of a current and a magnitude of electric resistance vary with a target temperature, a proportion of the conductive material 44 to the nonconductive material 42, and a magnitude of electric resistance of the conductive material 44.

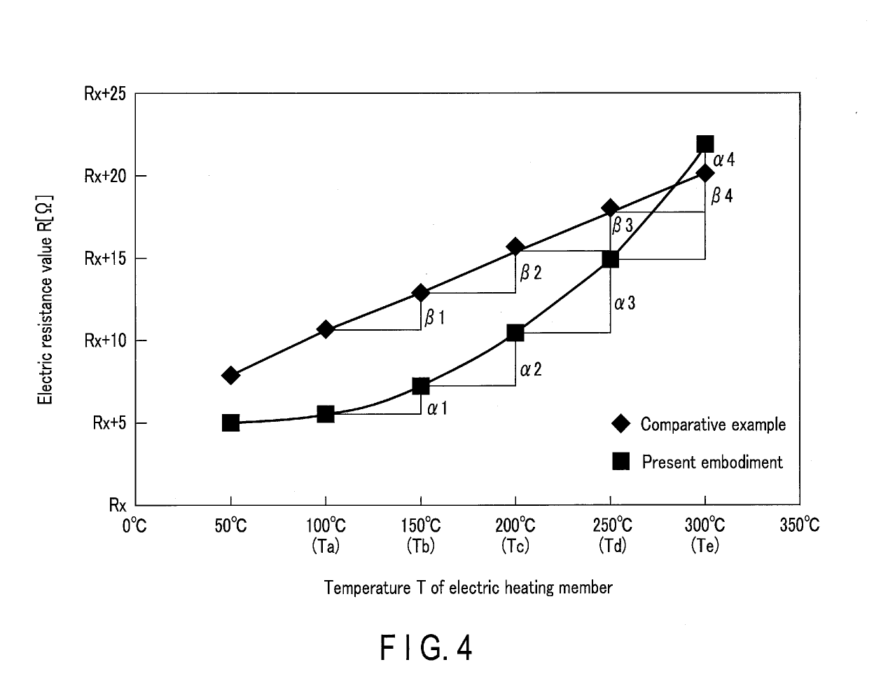

Now, regarding the above-described electric heating member 32, a relationship between a temperature T and an electric resistance value R of the electric heating member 32 as shown in FIG. 4, for example, was obtained in an experiment performed between the one end 32a and the other end 32b for a case where there is provided a certain content of the conductive material 44 using silver in the nonconductive material 42 using ceramic coating material. In FIG. 4, a sensor not shown measures the temperature T of the electric heating member 32. Then, the electric resistance value R is measured every time a temperature of the electric heating member 32 is raised by 50° C. It is noted that on an axis of ordinate indicating the electric resistance value R in FIG. 4, a tick mark is put at each portion where a certain electric resistance value Rx is incremented by 5[Ω] from a lower side to an upper side.

A relationship between the temperature T and the electric resistance value R is as follows. In the electric heating member 32, the resistance value R of electric resistance between the one end 32a and the other end 32b changes in accordance with a change in the temperature T of the electric heating member 32. Particularly, in the electric heating member 32 according to the present embodiment, it is recognized that the electric resistance value R changes non-linearly as the temperature T rises. Assume that an inclination of the electric resistance value R from a temperature Ta (100° C.) to a temperature Tb (150° C.) is α1. Assume that an inclination of the electric resistance value R from the temperature Tb to a temperature Tc (200° C.) is α2. Assume that an inclination of the electric resistance value R from the temperature Tc to a temperature Td (250° C.) is α3. Assume that an inclination of the electric resistance value R from the temperature Td to a temperature Te (300° C.) is α4. In this case, the inclination α2 is greater than the inclination α1, the inclination α3 is greater than the inclination α2, and the inclination α4 is greater than the inclination α3. Additionally, it can be said that the inclination α4 is several times as great as the inclination α1 in comparison between the inclinations α1 and α4. It can be also said that the inclination α4 is several times as great as an inclination β4 in a comparative example which will be later described.

Accordingly, the electric resistance value R increases to a greater extent during a rise of 50° C. from the temperature Tb to the temperature Tc, than that during a rise of 50° C. from the temperature Ta to the temperature Tb. The electric resistance value R increases to a greater extent during a rise of 50° C. from the temperature Tc to the temperature Td, than that during a rise from the temperature Tb to the temperature Tc. The electric resistance value R increases to a greater extent during a rise of 50° C. from the temperature Td to the temperature Te, than that during a rise from the temperature Tc to the temperature Td. In the other words, in the electric heating member 32 according to the present embodiment, an amount of change in the resistance value R per unit temperature in a high-temperature state is larger than that in a low-temperature state. An amount of change in the resistance value R of electric resistance between the one end 32a and the other end 32b of the electric heating member 32, in accordance with a change in the temperature T of the electric heating member 32, increases as the temperature T rises. Thus, the higher the temperature T becomes, the more precisely the temperature T corresponding to the electric resistance value R as measured is estimated. Consequently, in the electric heating member 32 according to the present embodiment, the higher a temperature becomes, the more accurately the temperature T of the electric heating member 32 can be controlled based on the electric resistance value R. A user can adjust and control the electric resistance value R which serves as a target control value by adjusting a level of energy such as a current which is fed to the electric heating member 32, for example, to thereby control the electric heating member 32 at the desired temperature T. It is noted that in FIG. 4, an amount of change in a resistance value of the electric resistance R between the one end 32a and the other end 32b of the electric heating member 32, in accordance with a change in the temperature T of the electric heating member 32, is larger than an amount of change at an ordinary temperature (room temperature), at a target control temperature which is higher than an ordinary temperature (room temperature).

In the electric heating member 32 according to the present embodiment, when temperatures around 300° C. are referred, an amount of a change in the resistance value. R in accordance with a change in the temperature T is particularly large at the foregoing temperatures. Accordingly, the temperature T of approximately 300° C. which is considered to be a temperature suitable for coagulation or incision of living-body tissue, for example, is precisely estimated and controlled by measurement of the electric resistance value R between the terminals 32a and 32b of the electric heating member 32. In this manner, in the electric heating member 32 according to the present embodiment, the higher a temperature becomes, the more accurately a user is allowed to grasp the temperature T corresponding to the electric resistance value R. The electric heating member 32 according to the present embodiment can cope with a demand for precise control of the temperature T to approximately 300° C. which is supposed to be suitable for a user's treatment of an object being treated, by adjusting a magnitude of a current, for example.

For the electric heating member 32 according to the present embodiment, various mixing proportions for mixing the conductive material 44 into the nonconductive material 42 are combined and examined, and an example of a mixing proportion which provides a high temperature coefficient of resistance and high specific volume resistivity is derived. Thus, the electric heating member 32 according to the present embodiment can be formed by mixture of the conductive material 44 of an appropriate material into the nonconductive material 42 of an appropriate material in an appropriate mixing proportion, to be used as a heating element, and it is designed such that the temperature T corresponding to the electric resistance value R can be recognized by measurement of the electric resistance value R between the terminals 32a and 32b of the electric heating member 32. Accordingly, the temperature T of the electric heating member 32 can be accurately controlled based on the electric resistance value R.

Further, in the electric heating member 32 according to the present embodiment, the inclination α4 at temperatures around 300° C. which is supposed to be suitable for treatment can be made great. Thus, the electric heating member 32 according to the present embodiment can easily control the temperature T to approximately 300° C. which is supposed to be a desired temperature, based on the electric resistance value R.

It is noted that although the inclination α3 between 200° C. and 250° C. is smaller than the inclination α4, the inclination α3 can possibly be a few times as great as the inclination α1, for example. Thus, the temperature T of the electric heating member 32 can be accurately controlled not only at approximately 300° C., but also at temperatures between 200° C. and 300° C. which are supposed to be suitable for coagulation and incision of living-body tissue, in the same manner as described above.

Now, a comparative example shown in FIG. 4 will be described. In the comparative example, unlike the present embodiment, the nonconductive material 42 is not used, and stainless steel is formed in a shape of a thin film and is made long and sinuous.

A relationship between the temperature T and the electric resistance value R in the comparative example is as follows. Assume that an inclination of the electric resistance value R from a temperature Ta (100° C.) to a temperature Tb (150° C.) is β1. Assume that an inclination of the electric resistance value R from the temperature Tb to a temperature Tc (200° C.) is β2. Assume that an inclination of the electric resistance value R from the temperature Tc to a temperature Td (250° C.) is β3. Assume that an inclination of the electric resistance value R from the temperature Td to a temperature Te (300° C.) is β4. In this case, the inclinations β1, β2, β3, and β4 are substantially identical to one another. Accordingly, the electric resistance value R increases to substantially the same extent between the temperatures Ta and Tb, between the temperatures Tb and Tc, between the temperatures Tc and Td, and between the temperatures Td and Te. Thus, irrespective of whether a low-temperature state (a temperature which is low for treating living-body tissue using transmitted heat) or a high-temperature state (a temperature which is suitable for treating living-body tissue using transmitted heat (approximately 300° C., for example)) is provided, an amount of change in the electric resistance value R per unit temperature is substantially the same. In the electric heating member in the comparative example, the temperature T provided when the electric resistance value R is measured can be estimated. Nonetheless, in a case where a user desires to precisely control a temperature of the electric heating member in the comparative example, it can be said that the electric heating member in the comparative example is poorer in performance than the electric heating member 32 according to the present embodiment. Also, in the comparative example, since the electric heating member is formed in a shape of a thin film and is also formed in a long and sinuous shape, a strength of the electric heating member is reduced.

As shown in FIGS. 2A and 2B, the heat transmission body 34 of the treatment section 24 according to the present embodiment includes a treatment surface 34a which is configured to be brought into contact with living-body tissue and is configured to treat the living-body tissue, and a heat transmission surface 34b in which the electric heating member 32 is formed. As shown in FIG. 2A, the electric heating member 32 may be formed directly on the heat transmission surface 34b of the heat transmission body 34. In this case, a nonconductive material such as a ceramic material is used for the heat transmission body 34. As shown in FIG. 2B, the electric heating member 32 may be formed on the heat transmission surface 34b of the heat transmission body 34 with the insulation layer 36 being interposed therebetween. In this case, it is preferable that the heat transmission body 34 is formed of a material having good thermal conductivity such as an aluminum-alloy material or a copper-alloy material, for example. An example in which the insulation layer 36 is provided between the electric heating member 32 and the heat transmission body 34 (refer to FIG. 2B) will be mainly described here. It is preferable to use the same material as that of the nonconductive material 42 of the electric heating member 32, for the insulation layer 36. It is preferable that the insulation layer 36 is formed of a ceramic material (ceramic coating material). In this case, the insulation layer 36 can prevent a current from flowing through the heat transmission body 34 such as an aluminum-alloy material, for example, when a current is fed to the electric heating member 32. Additionally, it is preferable that a thickness of the insulation layer 36 is minimized in order to reduce loss in heat transmission from the electric heating member 32 to the heat transmission body 34.

The electric heating member 32 is formed on the heat transmission body 34 as shown in FIG. 2A, or on the insulation layer 36 as shown in FIG. 2B, without a need of being formed in a shape of a fine wire and in a sinuous shape. Accordingly, the electric heating member 32 according to the present embodiment is more easily manufactured than that in a case where stainless steel is used as described in the comparative example. Also, the electric heating member 32 is not required to be formed in a shape of a thin film, and can be formed so as to have an appropriate thickness. Thus, a disconnection in the electric heating member 32 can be suppressed.

The electric heating member 32 according to the present embodiment is formed by a method in which a substance resulted from mixture of silver powder into ceramic coating material in an appropriate proportion is coated onto the insulation layer 36 having an electrically-insulating property, for example. In the present embodiment, the electric heating member 32 is formed in an almost U shape including the one end 32a and the other end 32b.

In the electric heating member 32, the terminals 32a and 32b are not necessarily required to be arranged side by side. For example, as shown in FIG. 3B, in a case where the electric heating member 32 is formed in not an almost U shape, but a shape of an almost rectangle, it is preferable that the terminals 32a and 32b are placed in positions which are diagonally opposite to each other, or in positions which are separated from each other along a lengthwise direction as indicated by broken lines in FIG. 3B. As described, it is allowable to form the electric heating member 32 in various shapes.

As shown in FIG. 5A, the treatment section 24 according to the present embodiment is formed in a shape of a surgical knife (or a shape of a spatula, for example). With reference to a cross section shown in FIG. 5B, the treatment section 24 includes the electric heating member 32, the insulation layer 36, and the heat transmission body 34 which are sequentially arranged in the stated order from an inner side to an outer side. In the present embodiment, the electric heating member 32 in an almost U shape shown in FIG. 3A is used. As shown in FIG. 5B, in a cross section taken along a line 5B-5B in FIG. 5A, the electric heating member 32 is divided into two parts by the insulation layer 36. Accordingly, a route along which a current I is applied to the electric heating member 32 is determined. Then, the electric heating member 32 is interposed between a pair of metal plates 37a and 37b included in the heat transmission body 34. It is noted that as shown in FIG. 5A, when the current I is fed in a manner indicated by a broken line, resistance heating is caused in an inner part of the electric heating member 32 of the treatment section 24.

As shown in FIG. 5B, the treatment surface 34a of the heat transmission body 34 includes a planar area 38a which is suitable to be pressed against living-body tissue for coagulation, and an edge-like area 38b which is suitable for incision of living-body tissue. Additionally, the treatment section 24 is formed in an extremely small size such a size with a total length in a range between approximately several millimeters and a dozen or so millimeters and a total width in a range between approximately several millimeters and ten millimeters, for example, because the treatment section 24 is inserted into a body cavity, for example.

As shown in FIG. 1, the energy source 14 includes a controller 62 such a single or more processor which exercises various controls, an output device 64 which is configured to adjust and output energy (a current, for example) which is to be transmitted to the electric heating member 32, a detector 66 which is configured to measure electric resistance between the one end 32a and the other end 32b of the electric heating member 32, a memory 68, and an input device 70, and a display 72. The input device 70 is used by a user in making appropriate settings for the output device 64, the memory 68, the display 72, and the like. Also, the input device 70 is used for setting the temperature (target temperature) T of the electric heating member 32. The input device 70 may be designed so as to accept direct input of the target temperature T, or to allow stepless adjustment of the target temperature T using a lever, for example, and various types of input devices can be used as the input device 70. As for a type with a lever, it is preferable to put a tick mark for the temperature T of a desired control temperature (approximately 300° C. in the present embodiment) on the input device 70. It is preferable that the display 72 is placed in the treatment instrument 12.

The energy source 14 is connected with a switch 74 such as a foot switch or a hand switch configured to switch an output of the output device 64 between ON and OFF. The switch 74 may be placed in the treatment instrument 12, or may be connected with the energy source 14. Also, it is preferable that the switch 74 is placed in the treatment instrument 12 and is connected with the energy source 14.

Then, a relationship shown in FIG. 4, between the temperature T and the electric resistance value R of the electric heating member 32 according to the present embodiment, is stored in the memory 68 of the treatment system 10. On the other hand, the treatment system 10 can detect electric resistance which is provided when a current is fed between the one end 32a and the other end 32b of the electric heating member 32, as a measured value, with the detector 66. Accordingly, in the treatment system 10, the controller 62 can recognize the temperature T corresponding to the electric resistance value R read out from the memory 68 when the controller 62 recognizes a detection result provided by the detector 66. In other words, the controller (determination device) 62 reads out memory contents in the memory 68 in accordance with a detection result (measured electric resistance value. R) provided from the detector 66, and determines the temperature T of the electric heating member 32 at the present time. In the treatment system 10, the controller 62 can display the target temperature T, the measured electric resistance value R, and the temperature T at the present time (estimated temperature T of the electric heating member 32) which is determined in the controller (determination device) 62, on the display 72.

Next, operations of the treatment system 10 according to the present embodiment will be described.

In the memory 68, a relationship (refer to FIG. 4) between the temperature T of the electric heating member 32 of the treatment instrument 12 according to the present embodiment and the electric resistance value R between the terminals 32a and 32b of the electric heating member 32, is previously stored.

A user (operator) appropriately operates the input device 70, to set the target temperature T (300° C., for example). The user appropriately operates the input device 70, to appropriately set an output state (a time period taken for a temperature of the electric heating member 32 to reach the target temperature T, or the like) of the output device 64 at a time when the switch 74 is turned ON. Information which is input with the input device 70 is stored in the memory 68.

While the user brings the planar area 38a or the edge-like area 38b of the treatment section 24 of the treatment instrument 12, close to, or in contact with, an object being treated in living-body tissue, the user turns ON the switch 74. At that time, the controller 62 causes energy to be output (a current to be fed) to the terminals 32a and 32b of the electric heating member 32 from the output device 64, based on information which is input with the input device 70 and stored in the memory 68.

The detector 66 detects the electric resistance value R between the terminals 32a and 32b of the electric heating member 32 continuously, or at appropriate time intervals of several milliseconds or the like. The controller 62 reads out the temperature T corresponding to the measured electric resistance value R at that time from the memory 68, and displays the temperature T on the display 72. That is, in the treatment system 10 according to the present embodiment, the temperature T of the electric heating member 32 in a state where the switch 74 is pressed, is displayed on the display 72 by measurement of the electric resistance value R between the terminals 32a and 32b of the electric heating member 32. At that time, on the display 72, both of the electric resistance value R and the target temperature T at the present time may be displayed together with the temperature T, or only the temperature T at the present time may be displayed together with the target temperature T.

Then, the treatment system 10 according to the present embodiment keeps controlling an amount of energy output from the output device 64 under control of the controller 62, and makes the measured electric resistance value R identical to the electric resistance value R corresponding to the target temperature T stored in the memory 68. If the measured electric resistance value R is identical to the resistance value R corresponding to the target temperature T read out from the memory 68, it can be said that a temperature of the electric heating member 32 is identical or substantially identical to the target temperature T.

Heat of the electric heating member 32 at a temperature which is identical or substantially identical to the target temperature T is transmitted to the heat transmission body 34. Accordingly, with the area 38a or the area 38b of the treatment surface 34a, living-body tissue is appropriately treated.

To stop output of energy from the output device 64 after treatment is finished, the user turns OFF the switch 74.

As described above, according to the present embodiment, the following can be said.

The electric heating member 32 of the treatment section 24 of the treatment instrument 12 in the present embodiment is formed as a composite member by a method in which the nonconductive material 42 such as ceramic coating material is mixed with the conductive material 44 such as silver. Although the electric heating member 32 according to the present embodiment includes the conductive material 44 which provides extremely small specific volume resistivity as compared to that of a nichrome wire, the electric heating member 32 is formed as a composite member including not only the conductive material 44 but also the nonconductive material 42. Therefore, it is possible to improve the volume resistivity of the electric heating member 32 to a level close to the volume resistivity of the nichrome wire, for example. Accordingly, it is not necessary to form the electric heating member 32 in a fine, long, and sinuous shape, so that appropriate strength of the electric heating member 32 can be maintained and a disconnection can be prevented. Therefore, according to the present embodiment, the treatment instrument 12 which is easy to manufacture, is easy to maintain strength thereof, and can treat a treatment object using heat generated in the treatment section 24, can be provided.

As described above, the treatment section 24 is formed in an extremely small size such a size with a total length in a range between approximately several millimeters and a dozen or so millimeters and a total width in a range between several millimeters and ten millimeters because the treatment section 24 is inserted into a body cavity, for example. Thus, it is difficult to place a temperature sensor which is configured to measure a temperature of the electric heating member 32 in the treatment section 24, together with the electric heating member 32, and to measure the temperature T. In a case where the electric heating member 32 according to the present embodiment is used, the electric resistance value R between the terminals 32a and 32b exhibits behaviors shown in FIG. 4, in accordance with the temperature T. Thus, it is possible to grasp the temperature T of the electric heating member 32 by measuring the electric resistance value R without placing a temperature sensor in the electric heating member 32 for the user.

The higher a temperature of the electric heating member 32 becomes, the more accurately the user is allowed to grasp the temperature T corresponding to the measured electric resistance value R, of the electric heating member 32.

Now, it is assumed in the present embodiment that the temperature T at which living-body tissue is treated using transmitted heat is approximately 300° C. When the temperature T of the electric heating member 32 is controlled at approximately 300° C., the electric heating member 32 according to the present embodiment allows more accurate recognition of a temperature of the electric heating member 32 than when a temperature of the electric heating member 32 is lower than the temperature T, using the measured electric resistance value R. Accordingly, by appropriately controlling the electric resistance value R, it is possible to accurately control the temperature T at which living-body tissue is treated using transmitted heat, with the electric heating member 32.

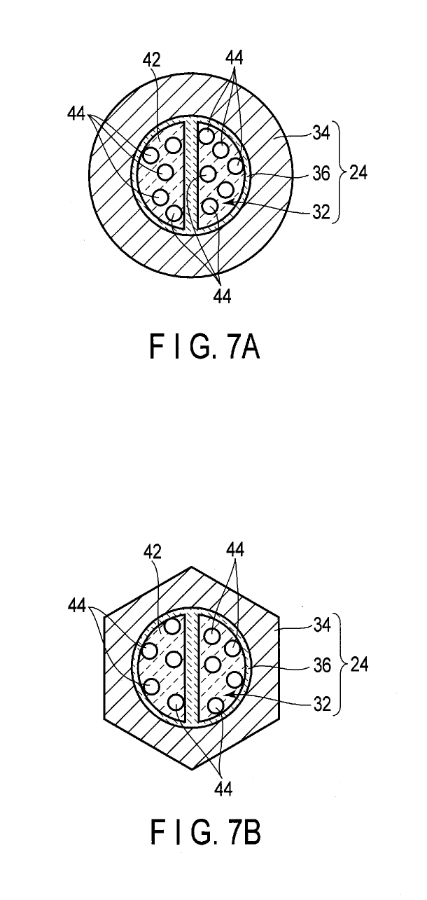

The electric heating member 32 shown in FIG. 5B has been described as having a substantially rectangular cross section. The electric heating member 32 according to the present embodiment can be formed in a shape of a columnar rod, a shape of a prismatic rod, or the like. Accordingly, although the treatment section 24 in FIG. 5A has been described as including a flat portion denoted by a reference numeral 38a and an edged portion denoted by a reference numeral 38b, the electric heating member 32 may be formed in a shape of a columnar rod and the heat transmission body 34 may be formed in a shape of a cylinder with which an outer face of the electric heating member 32 is covered (refer to FIG. 7A).

### Second Embodiment

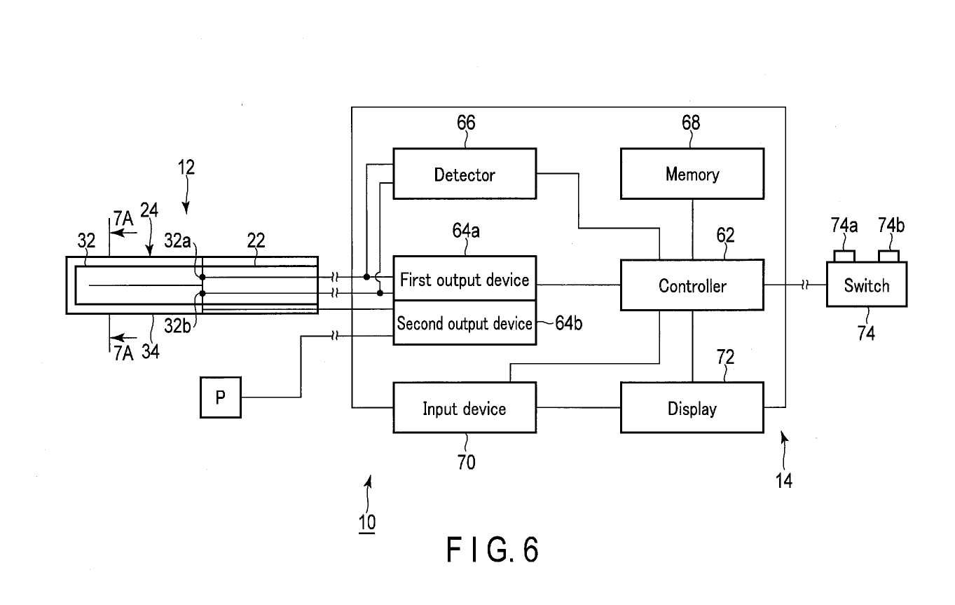

Next, a second embodiment will be described with reference to FIGS. 6 to 7B. In the present embodiment, which is a modification of the first embodiment, the same members or members having the same functions as those described in the first embodiment will be denoted by the same reference numerals, and detailed description thereof will be omitted.

As shown in FIG. 6, in the present embodiment, an energy source 14 includes first and second output devices 64a and 64b. In this case, the first output device 64a is used in feeding a current between one end 32a and the other end 32b of an electric heating member 32 and causing the electric heating member 32 to generate heat. The second output device 64b is used in feeding a high-frequency current to a heat transmission body 34 which is used as a high-frequency electrode. The second output device 64b is connected with a patient plate P. Accordingly, in the present embodiment, the treatment system 10 can provide monopolar high-frequency treatment.

As shown in FIG. 7A, a treatment section 24 is formed in a columnar shape, for example. The treatment section 24 includes the electric heating member 32, an insulation layer 36, and the heat transmission body 34 which are sequentially arranged in the stated order from an inner side toward an outer side. In FIG. 7A, the insulation layer 36 is formed between parts of the electric heating member 32, and the electric heating member 32 is formed in an almost U shape. Accordingly, it is possible to cause resistance heating (heat generation) in the electric heating member 32 by feeding a current to the electric heating member 32. The electric heating member 32 and the heat transmission body 34 are electrically insulated from each other.

In this embodiment, a switch 74 includes a first switch 74a for switching the first output device 64a between ON and OFF, and a second switch 74b for switching the first and second output devices 64a and 64b between ON and OFF.

Upon a switching operation of the first switch 74a, energy is output from the first output device 64a to the electric heating member 32 in the same manner as described in the first embodiment. At that time, by causing the electric heating member 32 to generate heat, appropriately controlling a temperature T, and transmitting the heat to the heat transmission body 34, it is possible to provide treatment such as coagulation or incision of living-body tissue.

When energy is output from the second output device 64b upon a switching operation of the second switch 74b, with the patient plate P being put on a patient, a current density of a portion in contact with the heat transmission body 34 in living-body tissue is increased, so that monopolar high-frequency treatment such as coagulation or incision of living-body tissue can be provided. Then, in the present embodiment, the electric heating member 32 and the heat transmission body 34 are electrically insulated from each other. Thus, energy can be output from the first and second output devices 64a and 64b at the same time. Therefore, upon a switching operation of the second switch 74b, monopolar high-frequency treatment can be provided, and treatment such as coagulation and incision of living-body tissue can be provided using heat transmitted to the heat transmission body 34.

It is noted that although the description has been made assuming that the treatment section 24 is in a columnar shape as shown in FIG. 7A in the present embodiment, the treatment section 24 may be in such a shape as shown in FIG. 5A, and it is preferable that the treatment section 24 is formed in a shape of a hexagonal prism shown in FIG. 7B. That is, it is allowable to form the treatment section 24 in various shapes. Additionally, in FIG. 7B, the insulation layer 36 is formed between parts of the electric heating member 32 and the electric heating member 32 is formed in an almost U shape. Accordingly, it is possible to cause resistance heating (heat generation) in the electric heating member 32 by feeding a current to the electric heating member 32.

### Third Embodiment

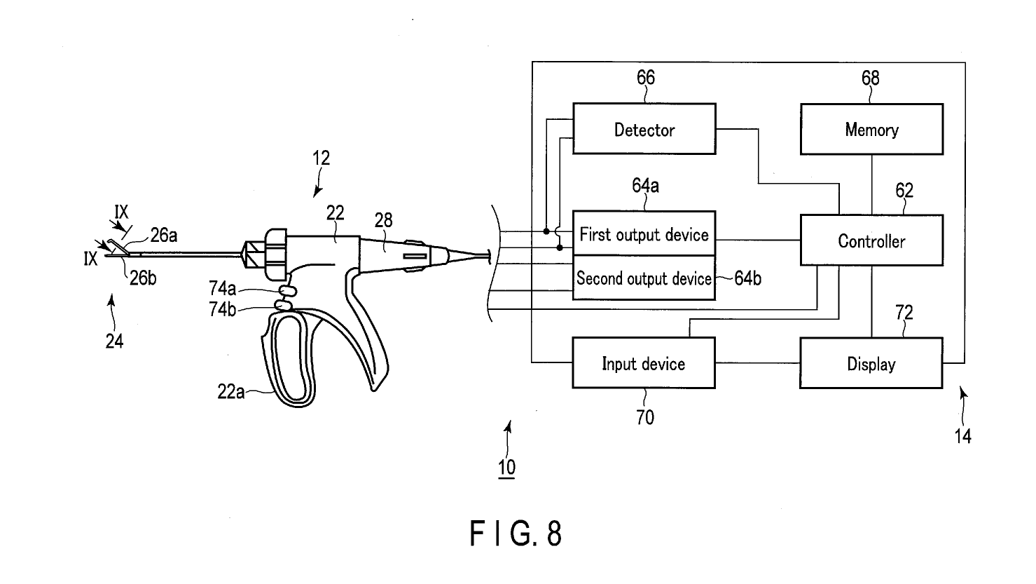

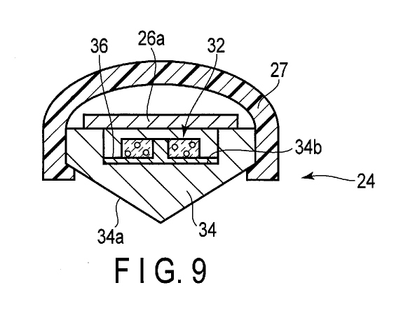

Next, a third embodiment will be described with reference to FIGS. 8 and 9. In the present embodiment, which is a modification of the first and second embodiments, the same members or members having the same functions as those described in the first and second embodiments will be denoted by the same reference numerals, and detailed description thereof will be omitted.

As shown in FIG. 8, in the present embodiment, a treatment section 24 includes a pair of grasping pieces 26a and 26b which can be opened and closed relative to each other.

In one or both, in other words, at least one, of the pair of grasping pieces 26a and 26b, an electric heating member (heating element) 32 which generates heat when a current is fed thereto is placed. An example in which the electric heating member 32 of the treatment section 24 of a treatment instrument 12 is placed in at least one of the pair of grasping pieces 26a and 26b will be described here. In the treatment section 24, only one grasping piece 26a swings upon an operation being performed on a handle 22a in a housing 22. In the one grasping piece 26a which swings, the electric heating member 32, an insulation layer 36, and a heat transmission body 34 are placed. In the present embodiment, the other grasping piece 26b is used as a top end of a vibration transmission member. A base end of the vibration transmission member is provided in the housing 22 and is attached to an ultrasonic vibrator unit 28.

As shown in FIG. 9, in the one grasping piece 26a out of the pair of grasping pieces 26a and 26b, the electric heating member 32, the heat transmission body 34, and the insulation layer 36 are placed. It is noted that the grasping piece 26a includes a cover 27 which is heat-resistant and has an electrically-insulating property. The electric heating member 32 and a portion not included in a treatment surface 34a in the heat transmission body 34 are covered with the cover 27. It is noted that although an example in which the treatment surface 34a is formed in an almost V shape is shown in FIG. 9, the treatment surface 34a may be in the other shapes such as a flat shape, for example.

It is preferable that the other grasping piece 26b is formed of a titanium-alloy material, for example. The other grasping piece 26b may be formed in a shape of a solid rod, and it is also preferable that the other grasping piece 26b includes the electric heating member 32, the insulation layer 36, and the heat transmission body 34 which are sequentially arranged in the stated order from an inner side toward an outer side as shown in FIG. 7A or 7B which has been referred to in the second embodiment.

When a first switch 74a is pressed with living-body tissue being interposed between the treatment surface 34a of the heat transmission body 34 of the one grasping piece 26a and the treatment surface 34a of the heat transmission body 34 of the other grasping piece 26b, a current flows from the first output device 64a through the electric heating member 32 of the first grasping piece 26a and the electric heating member 32 of the second grasping piece 26b, so that the respective electric heating members 32 are caused to generate heat. Then, heat (thermal energy) of the electric heating member 32 of the first grasping piece 26a is transmitted to the heat transmission body 34 via the insulation layer 36, and heat (thermal energy) of the electric heating member 32 of the second grasping piece 26b is transmitted to the heat transmission body 34 via the insulation layer 36. Thus, it is possible to provide treatment such as coagulation or incision to the living-body tissue which is interposed between the pair of grasping pieces 26a and 26b, using transmitted heat, at the target temperature T.

When a second switch 74b is pressed with living-body tissue being interposed between the treatment surface 34a of the heat transmission body 34 of the one grasping piece 26a and the treatment surface 34a of the heat transmission body 34 of the other grasping piece 26b, a current flows from the first output device 64a through the electric heating member 32 of the first grasping piece 26a and the electric heating member 32 of the second grasping piece 26b, so that the respective electric heating members 32 are caused to generate heat. Also, energy is output from the second output device 64b to the ultrasonic vibrator unit 28, and ultrasonic vibration is transmitted to the second grasping piece 26b. Thus, it is possible to provide treatment such as coagulation or incision to the living-body tissue which is interposed between the pair of grasping pieces 26a and 26b, using heat transmitted from the electric heating member 32 and transmitted ultrasonic vibration.

The heat transmission body 34 of each of the first grasping piece 26a and the second grasping piece 26b can be used also as a high-frequency electrode. In this case, it is possible to coagulate living-body tissue between the heat transmission body 34 of the one grasping piece 26a used as a high-frequency electrode and the heat transmission body 34 of the other grasping piece 26b used as a high-frequency electrode. Accordingly, in the present embodiment, bipolar high-frequency treatment can be provided.

In a case where treatment is provided using ultrasonic vibration, the pair of grasping pieces 26a and 26b should be configured such that only the one grasping piece 26a swings and can be opened and closed relative to the other grasping piece 26b. In a case where treatment using transmitted heat or bipolar high-frequency is provided without the use of ultrasonic vibration, the pair of grasping pieces 26a and 26b may be configured such that both of the grasping pieces 26a and 26b swing and can be opened and closed.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1.-13. (canceled)

14. A treatment instrument comprising:
a treatment section configured to treat living-body tissue; and
a grasping piece configured to support the treatment section and be gripped by an operator, wherein:
the treatment section includes;
an electric heating member which is formed by mixture of a conductive material into a nonconductive material, where resistance heating is caused in the electric heating member when a current is fed between one end and the other end of the electric heating member; and
a heat transmission body which includes:
a heat transmission surface configured to receive heat transmitted from the electric heating member; and
a treatment surface which is placed opposite to the heat transmission surface, the treatment surface being configured to treat the living-body tissue using the heat transmitted from the electric heating member,
a specific volume resistivity of the electric heating member is higher than that of the conductive material of the electric heating member and lower than that of the nonconductive material of the electric heating member, and
a temperature coefficient of resistance of the conductive material of the electric heating member is higher than that of a nichrome wire.

15. The treatment instrument according to claim 14, wherein a resistance value of electric resistance between the one end and the other end of the electric heating member is configured to change in accordance with a temperature change of the electric heating member of the treatment section.

16. The treatment instrument according to claim 14, wherein a resistance value of electric resistance between the one end and the other end of the electric heating member is configured to change non-linearly in accordance with a temperature change of the electric heating member of the treatment section.

17. The treatment instrument according to claim 16, wherein an amount of change in the resistance value of the electric resistance between the one end and the other end of the electric heating member in accordance with the temperature change of the electric heating member is configured to increase as a temperature rises.

18. The treatment instrument according to claim 16, wherein an amount of change in the resistance value of the electric resistance between the one end and the other end of the electric heating member in accordance with the temperature change of the electric heating member, is larger than an amount of change at a room temperature, at a target control temperature higher than the room temperature.

19. The treatment instrument according to claim 14, wherein:
the electric heating member includes a first terminal at the one end and a second terminal at the other end, and is formed in a substantially U shape, and
a clearance is provided between the one end and the other end.

20. The treatment instrument according to claim 19, wherein an insulation layer with an electrically insulating property is provided between the one end and the other end.

21. The treatment instrument according to claim 14, wherein:
a ceramic material is used for the nonconductive material, and
a metal material is used for the conductive material.

22. The treatment instrument according to claim 21, wherein the metal material is placed dispersedly in the ceramic material.

23. The treatment instrument according to claim 14, wherein:
the electric heating member and the heat transmission body are electrically insulated from each other, and
the heat transmission body is used as a high-frequency electrode.

24. The treatment instrument according to claim 14, wherein:
the treatment section includes a pair of grasping pieces configured to be opened and closed relative to each other, and
the electric heating member and the heat transmission body are placed in at least one of the pair of grasping pieces.

25. The treatment instrument according to claim 14, wherein the treatment section is formed in a shape of a surgical knife.

26. The treatment instrument according to claim 14, wherein the specific volume resistivity of the electric heating member is made close to, or higher than that of a nichrome wire.

27. A treatment instrument comprising an electric heating member which is formed by mixture of a conductive material into a nonconductive material, where resistance heating is caused in the electric heating member when a current is fed between one end and the other end of the electric heating member,
wherein:
a specific volume resistivity of the electric heating member is higher than that of the conductive material of the electric heating member and lower than that of the nonconductive material of the electric heating member,
a temperature coefficient of resistance of the conductive material of the electric heating member is higher than that of a nichrome wire, and
an inclination of a change in an electric resistance value of the electric heating member per unit temperature increases as a temperature of the electric heating member rises.

28. An end effector comprising:
an electric heating member which is formed by mixture of a conductive material into a nonconductive material, where resistance heating is caused in the electric heating member when a current is fed between one end and the other end of the electric heating member; and
a heat transmission body which includes:
a heat transmission surface configured to receive heat transmitted from the electric heating member; and
a treatment surface which is placed opposite to the heat transmission surface, the treatment surface being configured to treat the living-body tissue using the heat transmitted from the electric heating member,
wherein:
a specific volume resistivity of the electric heating member is higher than that of the conductive material of the electric heating member and lower than that of the nonconductive material of the electric heating member, and
a temperature coefficient of resistance of the conductive material of the electric heating member is higher than that of a nichrome wire.

29. A treatment system comprising:
the treatment instrument as recited in claim 14; and
an energy source configured to output energy to the treatment instrument.

30. The treatment system according to claim 29, wherein the energy source includes:
an output device configured to output energy to the electric heating member;
a detector configured to detect an electric resistance value of the electric heating member when the energy is output to the electric heating member from the output device;
a memory configured to store a relationship between an electric resistance value of the electric heating member and a temperature including a target temperature of the electric heating member, the electric resistance value of the electric heating member changing in accordance with the temperature; and
one or more processor configured to implement:
controlling output of the energy in the output device while controlling the electric resistance value of the electric heating member detected by the detector, and
conforming or substantially conforming the electric resistance value detected by the detector to the electric resistance value stored in the memory in accordance with the target temperature.
